(19) United States
(12) Patent Application Publication
(10) Pub. No.: US 2023/0414621 A1
(43) Pub. Date: Dec. 28, 2023

(54) NO-PDE5 INHIBITOR FOR USE IN TREATING DRY AGE-RELATED MACULAR DEGENERATION, GEOGRAPHIC ATROPHY AND GLAUCOMA-ASSOCIATED NEURODEGENERATION

(71) Applicant: Nicox S.A., Valbonne (FR)

(72) Inventors: Tomas NAVRATIL, Carrboro, NC (US); Francesco IMPAGNATIELLO, Milano (IT); Nicoletta ALMIRANTE, Milano (IT)

(21) Appl. No.: 18/251,283

(22) PCT Filed: Oct. 28, 2021
(86) PCT No.: PCT/EP2021/079958

§ 371 Date: May 1, 2023

(30) Foreign Application Priority Data
Nov. 2, 2020 (EP) 20205111.6

(51) Int. Cl. A61K 31/519; A61K 31/506

(52) U.S. Cl. CPC A61K 31/519; A61K 31/506

(57) ABSTRACT

The invention relates to the use of nitric oxide releasing cyclic guanosine 3′,5′ monophosphate (cGMP) phosphodiesterase type 5 inhibitors (NO-PDES inhibitors) in a method for the treatment of dry age-related macular degeneration and geographic atrophy. This invention also relates to the use of such compounds to provide neuroprotection to the eye in a patient suffering from glaucoma or retinal neuropathies.

The invention relates to the use of nitric oxide (NO)-releasing phosphodiesterase type 5 inhibitors (NO-PDE5 inhibitors) in a method for the treatment of dry age-related macular degeneration, geographic atrophy, and other ophthalmic neuropathies. This invention also relates to the use of such compounds to provide a neuroprotective effect to the eye in a patient suffering from glaucoma or retinal neuropathies.

Age-related macular degeneration (AMD) is a disease associated with aging that progressively destroys a person's central vision. AMD begins with characteristic drusen (yellow deposits) in the macula between the retinal pigment epithelium and the underlying choroid.

Age-related maculopathy may progress to two main forms of AMD: 1) wet-AMD and dry-AMD. Wet-AMD typically affects about 10% of total AMD patients and causes vision loss due to abnormal blood vessel growth in the choriocapillaries, through Bruch's membrane, ultimately leading to blood and protein leakage under the macula. Differently, dry-AMD affects the remaining 90% of patients, occurs when light-sensitive cells (photoreceptors) in the macula slowly break down, gradually causing vision loss in the affected eye. Dry-AMD can progress into intermediate or advanced stages of dry AMD such as geographic atrophy that is generally considered to be the non-wet end-stage of AMD.

Geographic atrophy is characterized by the presence of sharply demarcated atrophic lesions of the outer retina, retinal pigment epithelium (RPE) and underlying choriocapillaris (CC) that lead to photoreceptors death.

Pooled global prevalence (mapped to an age range between 45-85 years) of any age-related macular degeneration is 8.7% with the projected number of affected individuals estimated to grow to 196 million in 2020 and 288 million in 2040 (Wong et al., Lancet Glob Health. 2014;2(2):e106-e116).

Glaucoma is the leading cause of irreversible blindness worldwide, by the year 2040, an estimated 111 million people will have glaucoma, many of which will be bilaterally blind (Tham et al.: Global prevalence of glaucoma and projections of glaucoma burden through 2040: a systematic review and meta-analysis. Ophthalmology 2014; 121:2081-90). Elevated intraocular pressure (IOP)-related optic neuropathy characterized by degeneration of retinal ganglion cells (RGCs) and axons in the optic nerve is the major hallmark in advanced stage glaucoma. Clinical results indicate that significant and sustained TOP lowering slows vision loss in glaucoma patients; however, IOP lowering is not always effective in obviating damage to the nerves in glaucomatous conditions. Furthermore, lowering IOP in patients with ocular hypertension slows, but does not completely prevent the onset of glaucomatous disease.

The pathophysiological characteristics of dry age-related macular degeneration, geographic atrophy and glaucoma are different; however, these diseases are all characterized by neurodegenerative conditions such as photoreceptors atrophy, degeneration of retinal ganglion cells and axons leading to optic nerve atrophy with a prominent involvement of defects in ocular vascular reactivity. Therefore, neuroprotective therapies may prevent or retard the progression of these pathologies and of the onset of blindness associated with the course of the diseases.

Several studies and clinical trials have been performed to find therapies for dry-ADM; some examples of investigated drugs and therapies are: neuroprotective agents such as brimonidine and ciliary neurotrophic factor (CNTF); immune-modulators such as Lampalizumab and Zimura; suppressors of inflammation such as Iluvien; anti-oxidative stress agents such as Risuteganib (Luminate) and ocular gene therapy (Marcella Nebbioso et al; Int. J. Mol. Sci. 2019, 20, 1693).

US 2019/152967 (The Schepens Eye Research Institute, Inc) discloses the use of peroxisome proliferator-activated receptors-gamma (PPAR-gamma) selective agonists such as troglitazone for the treatment of late stage dry-AMD and GA. The reported data show that the PPAR-gamma-agonist troglitazone suppresses oxidized lipid-induced cell death in RPE that is associated with the development of dry AMD and GA.

The choroidal circulation provides nutrients to the photoreceptors and removes waste products from the retinal pigment epithelium (RPE).

Recent studies have hypothesized that abnormalities of the choroidal circulation and choriocapillaris may contribute to the progression of geographic atrophy.

Braun et al. (Invest Ophthalmol Vis Sci. 2019 Dec. 2; 60(15):4985-4990) reports data showing a significant correlation between dry-AMD stage and choriocapillaris perfusion, particularly for those in the peripheral regions of the macula, an effect typically not observed in the eyes of age-matched healthy controls.

Nitric oxide (NO) plays an important role in numerous vascular physiological processes including regulation of blood pressure and blood flow, platelet aggregation, and leukocyte adhesion (Moncada et al., 1991). In addition, NO, formed within the endothelial cells by endothelial nitric oxide synthase (eNOS) and perivascular nitrergic neurons by the neuronal nitric oxide synthase (nNOS), regulates choroidal blood flow.

Garcia et al. (J. Neurosci. Res. 90(3), 656-663 (2012)) administered neural-derived peptides Cop-1 and A91, liberating T-cells activated to Th2-type immune response, capable of counteracting NO production. This was seen when glial cells were cultured with these activated T-cells in vitro, leading to the reduced production of NO. Furthermore, the iNOS mRNA expression significantly diminished, iNOS being the most important enzyme in NO synthesis after neuronal injury.

Imran A. Bhutto et al. (Exp Eye Res. 2010 January; 90(1):155-167) discloses that in AMD eyes, the immunoreactivity for the constitutive NOS's (nNOS and eNOS) is significantly reduced in retina and choroid causing a reduction of NO endogenous neo-synthesis. This deficiency may ultimately result in vasoconstriction, ischemia of the choriocapillaris along with major hemodynamic changes supporting the hypothesis that reduced NO may play an important role in reducing blood flow and oxygenation of submacular choroid which is critical for central visual function.

Kim et al. (Acta Ophthalmol, 2013. 91, 183-188) discloses the results of a study that addresses choroidal and retinal vascular changes in healthy volunteers after oral sildenafil citrate, a phosphodiesterase type 5 (PDE5) inhibitor and potent vasodilator. In this and other reports, choroidal perfusion and thickness after systemic sildenafil dosing is significantly increased. Yiu et al. (Scientific Reports, 2019 9:5059) found that regardless of the AMD diagnosis, systemic sildenafil administered to old individuals results in 6.0% to 8.6% increase in choroidal thickness compared to untreated individuals.

Lauren K. Wareham et al., Neurobiol dis. 2019 January; 121:65-75 discloses the use of tadalafil and related compounds that enhance cGMP bioavailability as potential therapeutics for retinal ganglion cells (RGCs) neuroprotection.

In particular, the publication discloses the effects of orally administered tadalafil, a PDE5 inhibitor, in murine models of two forms of glaucoma, namely: primary open angle glaucoma (POAG) and primary angle-closure glaucoma (PACG); the results show that tadalafil prevented IOP-induced degeneration of retinal ganglion cells (RGCs) but did not alter iOP or mean arterial pressure. In addition, in vitro studies in primary purified RGCs indicated that high cGMP levels have the potential to mitigate both necrotic and apoptotic cell death in retina and, more specifically, of RGCs.

Additional studies have revealed neuroprotective properties of NO via activation of its downstream intracellular mediator cGMP and, other downstream effectors including protein kinases and Ca2+ channels.

US 2006/0014754 (Pfizer Inc.) discloses the systemic (oral or parenteral) administration of the PDE5-inhibitors and in particular of sildenafil citrate for the prevention or treatment of central retinal artery occlusion, central retinal vein occlusion and optic neuropathy including macular (dry) degeneration. US 2006/0014754 discloses a study that evaluate the effects of orally administered sildenafil citrate on optic nerve head blood flow and choroid blood flow using laser doppler flowmetry, however, no data are reported.

US 2002/0168424 discloses a topical drug for the treatment of glaucoma which comprises a mixture of a NO donor such as the nitrovasodilators minoxidil, nitroglycerin, L-arginine, isosorbide dinitrate or nitroprusside, and a phosphodiesterase type 5 (PDE5) inhibitor such as sildenafil citrate. This combination increases blood circulation to the optic nerve and has ocular hypotensive activity. US 2002/0168424 does not disclose experimental results.

WO 2017/085056 and WO 2018/215433 (Topadur Pharma AG) disclose dual-pharmacology NO-releasing PDE5 inhibitors that are potentially useful in a variety of therapeutic areas where a disturbed cGMP balance occurs and/or PDE inhibition is thought to be beneficial, in particular these compounds can have vasodilator, anti-vasospastic, anti-platelet, natriuretic and diuretic activities.

WO 2020/030489 (Nicox SA) discloses NO-releasing PDE5 inhibitors and their use for the treatment of ocular diseases associated with elevated intraocular pressure such as ocular hypertension and glaucoma, and for treating retinopathies. The results of the disclosed studies in animal models show that these NO-releasing phosphodiesterase type 5 inhibitors reduce intraocular pressure. However, in WO 2020/030489 no inference is made on a potential action of these compounds on choroidal blood flow and retinal oxygenation.

U.S. Pat. Nos. 10,195,140 and 10,456,356 in the name of Neurotech USA Inc, disclose a CNTF-secreting ophthalmic device encapsulated cell technology (NT-501 ECT) for the treatment of various kinds of ophthalmic disorders, including retinitis pigmentosa, geographic atrophy (dry age-related macular degeneration), glaucoma and/or macular telangiectasia The device contains ARPE-19 cells that are genetically engineered to secrete a therapeutically effective amount of ciliary neurotrophic factor (CNTF) and the device is placed in the pars plana of the eye through a surgical procedure.

Ciliary neurotrophic factor (CNTF) is a protein that is involved in promoting neurotransmitter synthesis and neurite outgrowth in neuronal populations. CNTF is a survival factor for neuronal cells, including neurons and oligodendrocytes, and has been demonstrated to have a protective role for photoreceptors.

NT-501 ECT is currently under clinical evaluation in particular, NT-501 ECT is in Phase 2 for glaucoma and in Phase 1 for ischemic optic neuropathy.

From the above, neurodegenerative eye diseases represent a burden with high social and economic impact worldwide, they affect a high number of patient that includes a considerable number of working-age adults. Few effective treatments currently exist in the clinic that delay the course of development of dry macular degeneration and geographic atrophy or that prevent or delay the progressive optic neuropathy associated with glaucoma.

Thus, a great need exists for treatments of dry age-related macular degeneration and geographic atrophy and for eye neuroprotective treatments for the clinical glaucoma management.

The present invention relates to the use of dual acting compounds of formula (I), formula (II) or formula (III) that show nitric oxide (NO)-releasing properties and phosphodiesterase type-5 (PDE5)-inhibitory activity, namely:NO-PDE5 inhibitors for treating dry age-related macular degeneration, geographic atrophy and or for preventing or inhibiting glaucoma-associated optic nerve neurodegeneration.

It is believed that the NO-PDE5 inhibitors of the invention ameliorate the choroidal circulation, in particular the NO-PDE5 inhibitors are able to ameliorate the blood flow in the choriocapillaris. The increase in blood flow may likely enhance tissue oxygenation and choroidal thickness and potentially delay or even prevent retinal pigment epithelium (RPE) degeneration and photoreceptor cell death as well as the occurrence of geographic atrophy associated with dry AMD. In addition, it is believed that the NO-PDE5 inhibitors of the invention ameliorate or prevent the death of the retinal ganglion cells and thus provide neuroprotective effect and, reduce or eliminate the progressive vision loss associated with the progression of the glaucoma disease.

The intravitreal injection (not via eye drop) of the NO-PDE5 inhibitors of the invention has the advantage that the therapeutic effect can be obtained with fewer adverse effects typically associated with the systemic (oral, parenteral, transdermal) administration of vasodilators such as the nitric oxide (NO) donors, nitroglycerin and isosorbide dinitrate or, of the PDE5 inhibitors. Examples of adverse effects associated with NO donors include, but are not limited to, headache, severe hypotension.

The invention relates to the use of a compound of formula (I), formula (II) or formula (III) (NO-PDE5 inhibitor) or a stereoisomer or a pharmaceutically acceptable salt thereof in a method of treating dry age-related macular degeneration and/or geographic atrophy, in a method of providing neuroprotection to a patient suffering from glaucoma, or in a method of treating or preventing retinal neuropathies

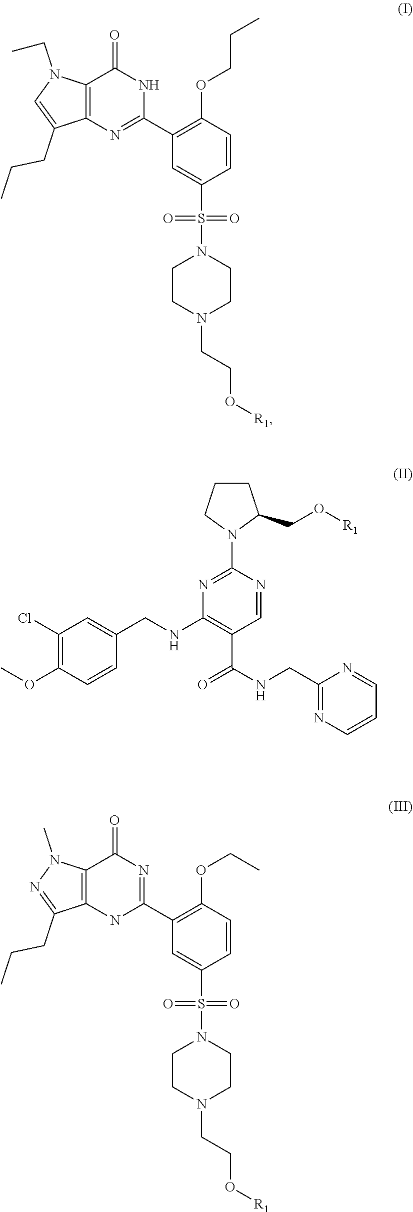

wherein:
R₁ is the residue of a nitric oxide releasing molecule having the following formula:
R₁=—C(O)—(O—CH₂)_{y}(CH₂)ₘ—[—(CH₂)ₙ]ₚ—(CH—ONO₂)_{q}—CH₂—ONO₂
wherein:
y is 1 or 0; preferably y is 0;
p is 1 or 0;
q is 1 or 0;
m is an integer ranging from 1 to 10; preferably m is from 1 to 6;
n is an integer ranging from 1 to 6; preferably n is 1 or 2;
   preferably in the compounds of formula (I), formula (II) or formula (III):
   when y=0, R₁ is selected from the group consisting of:

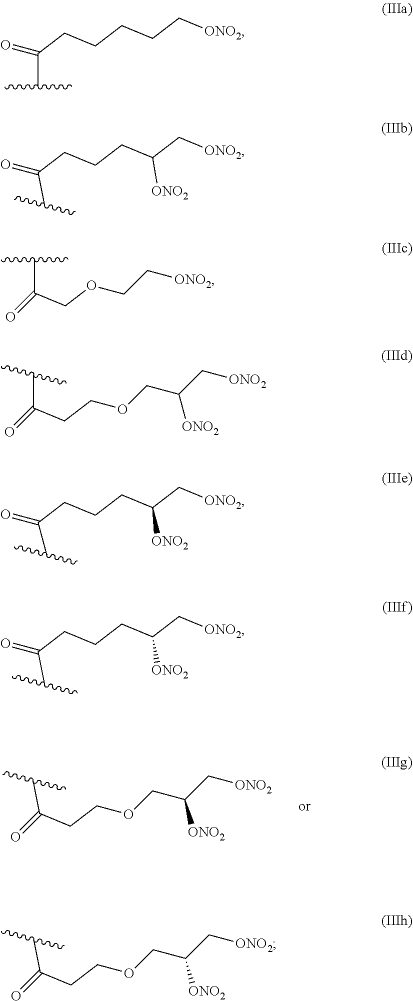

when y=1, R₁ is preferably selected from the group consisting of:

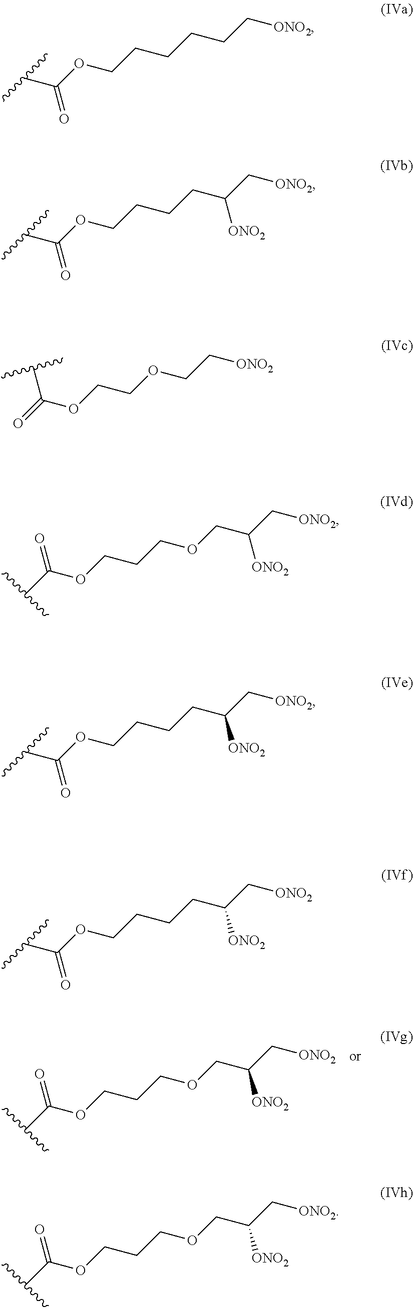

Another embodiment of the invention encompasses the use of a compound of formula (I) or a pharmaceutically acceptable salt thereof wherein y is 0 and R₁ is selected from the group consisting of the radicals (IIIa)-(IIIh) as defined above, preferably R₁ is selected from (IIIg) or (IIIh), most preferably R₁ is (IIIg) in a method of treating dry age-related macular degeneration and/or geographic atrophy.

Another embodiment of the invention provides the use of a compound of formula (II) or a pharmaceutically acceptable salt thereof wherein y is 0 and R₁ is selected from the group consisting of the radicals (IIIa)-(IIIh) as defined above, preferably R₁ is selected from (IIIa), (IIIg) or (IIIh), most preferably R₁ are (IIIa) or (IIIg) in a method of treating dry age-related macular degeneration and/or geographic atrophy.

Another embodiment of the invention encompasses the use of a compound of formula (III) or a pharmaceutically acceptable salt thereof wherein y is 0 and R₁ is selected from the group consisting of the radicals (IIIa)-(IIIh) as defined above, preferably R₁ is selected from (IIIa), (IIIg) or (IIIh), most preferably R₁ is (IIIa) in a method of treating dry age-related macular degeneration and/or geographic atrophy.

Another embodiment of the invention encompasses the use of a compound of formula (I) or a pharmaceutically acceptable salt thereof wherein y is 0 and R₁ is selected from the group consisting of the radicals (IIIa)-(IIIh) as defined above, preferably R₁ is selected from (IIIg) or (IIIh), most preferably R₁ is (IIIg) in a method of providing neuroprotection to a patient suffering from glaucoma.

Another embodiment of the invention provides the use of a compound of formula (II) or a pharmaceutically acceptable salt thereof wherein y is 0 and R₁ is selected from the group consisting of the radicals (IIIa)-(IIIh) as defined above, preferably R₁ is selected from (IIIa), (IIIg) or (IIIh), most preferably R₁ are (IIIa) or (IIIg) in a method of providing neuroprotection to patients suffering from glaucoma.

Another embodiment of the invention encompasses the use of a compound of formula (III) or a pharmaceutically acceptable salt thereof wherein y is 0 and R₁ is selected from the group consisting of the radicals (IIIa)-(IIIh) as defined above, preferably R₁ is selected from (IIIa), (IIIg) or (IIIh), most preferably R₁ is (IIIa) in a method of providing neuroprotection to a patient suffering from glaucoma.

Another embodiment of the invention relates to the use of a compound of formula (I) or a pharmaceutically acceptable salt thereof wherein y is 0 and R₁ is selected from the group consisting of the radicals (IIIa)-(IIIh) as defined above, preferably R₁ is selected from (IIIg) or (IIIh), most preferably R₁ is (IIIg) in method of treating or preventing retinal neuropathies.

Another embodiment of the invention provides the use of a compound of formula (II) or a pharmaceutically acceptable salt thereof wherein y is 0 and R₁ is selected from the group consisting of the radicals (IIIa)-(IIIh) as defined above, preferably R₁ is selected from (IIIa), (IIIg) or (IIIh), most preferably R₁ are (IIIa) or (IIIg) in a method of treating or preventing retinal neuropathies.

Another embodiment of the invention provides the use of a compound of formula (III) or a pharmaceutically acceptable salt thereof wherein y is 0 and R₁ is selected from the group consisting of the radicals (IIIa)-(IIIh) as defined above, preferably R₁ is selected from (IIIa), (IIIg) or (IIIh), most preferably R₁ is (IIIa) in a method of treating or preventing retinal neuropathies.

Another embodiment of the invention encompasses the use of a compound of formula (I) or a pharmaceutically acceptable salt thereof wherein y is 1 and R₁ is selected from the group consisting of the radicals (IVa)-(IVh) as defined above, preferably R₁ is selected from (IVa) or (IVe) in a method of treating dry age-related macular degeneration and/or geographic atrophy.

Another embodiment of the invention provides the use of a compound of formula (II) or a pharmaceutically acceptable salt thereof wherein y is 1 and R₁ is selected from the group consisting of the radicals (IVa)-(IVh) as defined above, preferably R₁ is selected from (IVa) or (IVe) in a method of treating dry age-related macular degeneration and/or geographic atrophy.

Another embodiment of the invention provides the use of a compound of formula (III) or a pharmaceutically acceptable salt thereof wherein y is 1 and R₁ is selected from the group consisting of the radicals (IVa)-(IVh) as defined above, preferably R₁ is selected from (IVa) or (IVe) in a method of treating dry age-related macular degeneration and/or geographic atrophy.

Another embodiment of the invention encompasses the use of a compound of formula (I) or a pharmaceutically acceptable salt thereof wherein y is 1 and R₁ is selected from the group consisting of the radicals (IVa)-(IVh) as defined above, preferably R₁ is selected from (IVa) or (IVe) in a method of providing neuroprotection to a patient suffering from glaucoma.

Another embodiment of the invention provides the use of a compound of formula (II) or a pharmaceutically acceptable salt thereof wherein y is 1 and R₁ is selected from the group consisting of the radicals (IVa)-(IVh) as defined above, preferably R₁ is selected from (IVa) or (IVe) in a method of providing neuroprotection to patients suffering from glaucoma.

Another embodiment of the invention provides the use of a compound of formula (III) or a pharmaceutically acceptable salt thereof wherein y is 1 and R₁ is selected from the group consisting of the radicals (IVa)-(IVh) as defined above, preferably R₁ is selected from (IVa) or (IVe) in a method of providing neuroprotection to patients suffering from glaucoma.

Another embodiment of the invention relates to the use of a compound of formula (I) or a pharmaceutically acceptable salt thereof wherein y is 1 and R₁ is selected from the group consisting of the radicals (IVa)-(IVh) as defined above, preferably R₁ is selected from (IVa) or (IVe) in method of treating or preventing retinal neuropathies.

Another embodiment of the invention provides the use of a compound of formula (II) or a pharmaceutically acceptable salt thereof wherein y is 1 and R₁ is selected from the group consisting of the radicals (IVa)-(IVh) as defined above, preferably R₁ is selected from (IVa) or (IVe) in a method of treating or preventing retinal neuropathies.

Another embodiment of the invention provides the use of a compound of formula (III) or a pharmaceutically acceptable salt thereof wherein y is 1 and R₁ is selected from the group consisting of the radicals (IVa)-(IVh) as defined above, preferably R₁ is selected from (IVa) or (IVe) in a method of treating or preventing retinal neuropathies.

Compounds of formula (I), formula (II) or formula (III) that can form salts can be used in the non-salt form or in the form of pharmaceutically acceptable salt. Suitable acids for use in the preparation of pharmaceutically acceptable salts include: citric acid, oxalic, malic, tartaric, succinic acid, acetic acid, propionic acid, lactic acid, hydrochloric acid hydrobromic acid, sulfuric acid, phosphoric acid, nitric acid, methanesulfonic acid, ethanesulfonic acid or benzenesulfonic acid.

Included within the scope of the present invention are the individual enantiomers of the compounds of formula (I), of formula (II), or of formula (III), as well as their diastereoisomers, racemic and non-racemic mixtures. As used herein, stereoisomer refers to enantiomers and diastereoisomers.

Another embodiment of the invention provides the use of a compound of formula (I) or a pharmaceutically acceptable salt thereof in a method of treating dry age-related macular degeneration or geographic atrophy, wherein said compound is selected from the group consisting of:

2-(4-(3-(5-ethyl-4-oxo-7-propyl-4,5-dihydro-3H-pyrrolo[3,2-d]pyrimidin-2-yl)-4-propoxyphenylsulfonyl)piperazin-1-yl)ethyl 3-[(2S)-2,3-bis(nitrooxy)propoxy]propanoate (Compound (1))

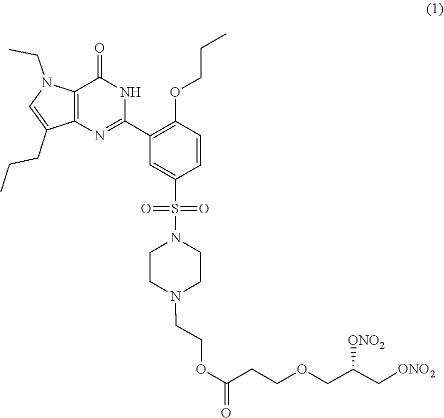

2-(4-((3-(5-ethyl-4-oxo-7-propyl-4,5-dihydro-3H-pyrrolo[3,2-d]pyrimidin-2-yl)-4-propoxyphenyl)sulfonyl)piperazin-1-yl)ethyl 2-(2-(nitrooxy)ethoxy)acetate (Compound (2))

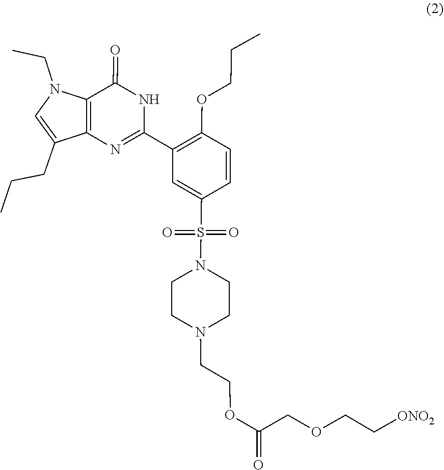

2-(4-(3-(5-ethyl-4-oxo-7-propyl-4,5-dihydro-3H-pyrrolo[3,2-d]pyrimidin-2-yl)-4-propoxyphenylsulfonyl)piperazin-1-yl)ethyl (5S)-5,6-bis(nitrooxy)hexanoate (Compound (3))

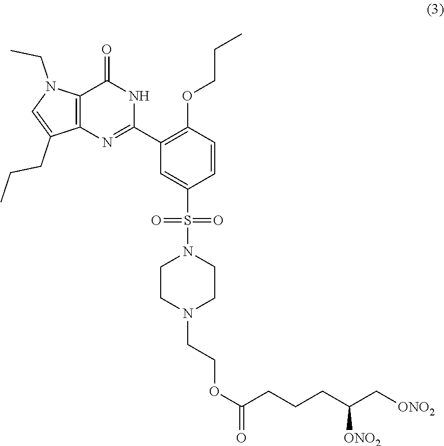

2-(4-((3-(5-ethyl-4-oxo-7-propyl-4,5-dihydro-3H-pyrrolo[3,2-d]pyrimidin-2-yl)-4-propoxyphenyl)sulfonyl)piperazin-1-yl)ethyl 6-(nitrooxy)hexanoate (Compound (4))

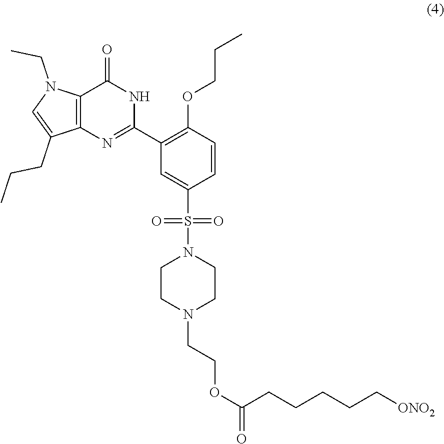

2-(4-(3-(5-ethyl-4-oxo-7-propyl-4,5-dihydro-3H-pyrrolo[3,2-d]pyrimidin-2-yl)-4-propoxyphenylsulfonyl)piperazin-1-yl)ethyl 6-(nitrooxy)hexyl carbonate (Compound (5))

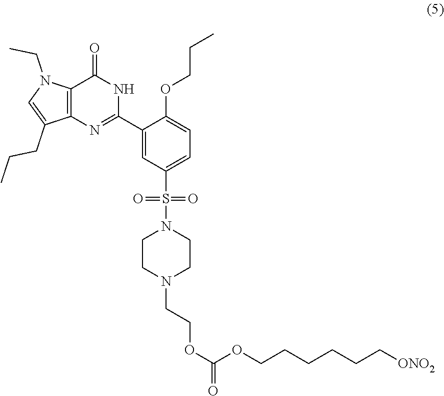

2-(4-(3-(5-ethyl-4-oxo-7-propyl-4,5-dihydro-3H-pyrrolo[3,2-d]pyrimidin-2-yl)-4-propoxyphenylsulfonyl)piperazin-1-yl)ethyl (5S)-5,6-bis (nitrooxy)hexyl carbonate (Compound (6))

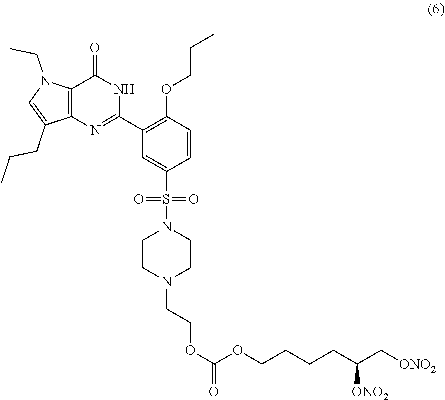

Another embodiment of the invention provides the use of a compound of formula (II) or a stereoisomer or a pharmaceutically acceptable salt thereof in a method of treating dry age-related macular degeneration and or geographic atrophy, wherein said compound is selected from the group consisting of:

[(2S)-1-(4-{[(3-chloro-4-methoxyphenyl)methyl]amino}-5-{[(pyrimidin-2-yl) methyl]carbamoyl}pyrimidin-2-yl)pyrrolidin-2-yl]methyl 6-(nitrooxy)hexanoate (Compound (7))

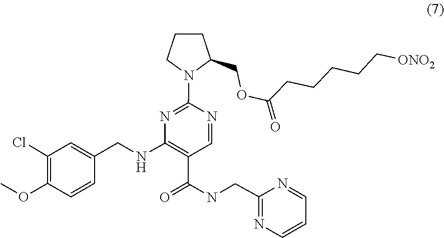

[(2S)-1-(4-{[(3-chloro-4-methoxyphenyl)methyl]amino}-5-{[(pyrimidin-2-yl) methyl]carbamoyl}pyrimidin-2-yl)pyrrolidin-2-yl]methyl (5S)-5,6-bis(nitrooxy) hexanoate (Compound (8))

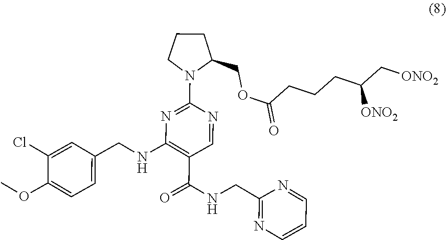

(S)-(1-(4-(3-chloro-4-methoxybenzylamino)-5-(pyrimidin-2-ylmethylcarbamoyl) pyrimidin-2-yl)pyrrolidin-2-yl)methyl 2-(2-(nitrooxy)ethoxy)acetate (Compound (9))

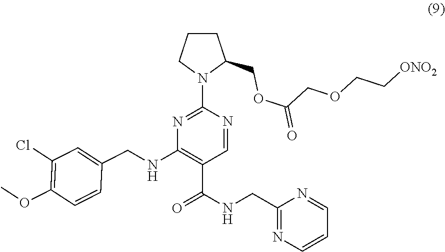

[(2S)-1-(4-{[(3-chloro-4-methoxyphenyl)methyl]amino}-5-{[(pyrimidin-2-yl) methyl]carbamoyl}pyrimidin-2-yl)pyrrolidin-2-yl]methyl 3-[(2S)-2,3-bis(nitrooxy) propoxy]propanoate (Compound (10))

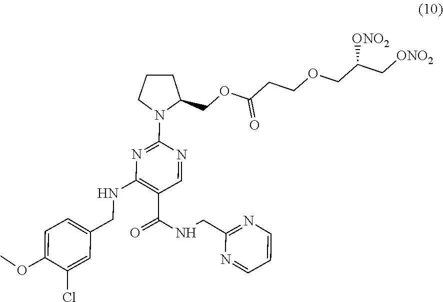

(S)-(1-(4-(3-chloro-4-methoxybenzylamino)-5-(pyrimidin-2-ylmethylcarbamoyl) pyrimidin-2-yl)pyrrolidin-2-yl)methyl 6-(nitrooxy)hexyl carbonate (Compound (11))

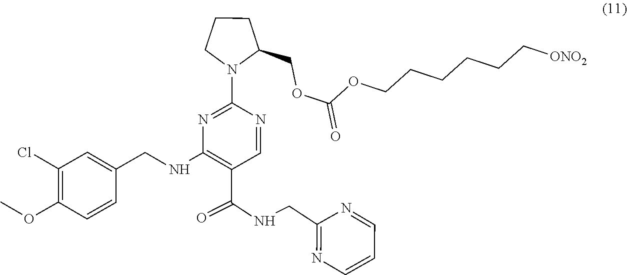

((S)-1-(4-(3-chloro-4-methoxybenzylamino)-5-(pyrimidin-2-ylmethylcarbamoyl) pyrimidin-2-yl)pyrrolidin-2-yl)methyl (5S)-5,6-bis(nitrooxy)hexyl carbonate (Compound (12))

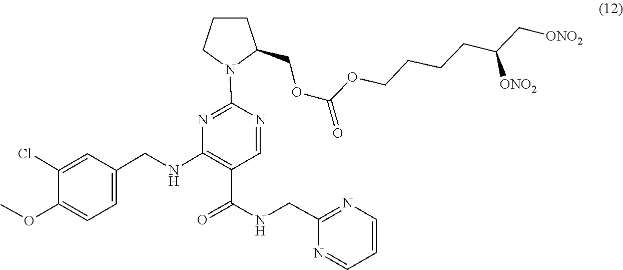

Another embodiment of the invention provides the use of a compound of formula (III) or a stereoisomer or a pharmaceutically acceptable salt thereof in a method of treating dry age-related macular degeneration and or geographic atrophy, wherein said compound is selected from the group consisting of:

2-{4-[4-ethoxy-3-(1-methyl-7-oxo-3-propyl-6,7-dihydro-1H-pyrazolo[4,3-d] pyrimidin-5-yl)benzene-1-sulfonyl]piperazin-1-yl}ethyl (5S)-5,6-bis (nitrooxy)hexanoate (Compound (13))

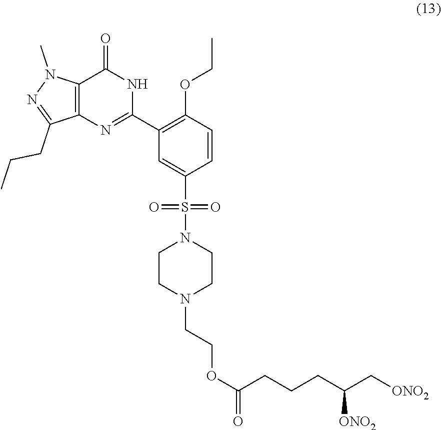

2-{4-[4-ethoxy-3-(1-methyl-7-oxo-3-propyl-6,7-dihydro-1H-pyrazolo [4,3-d] pyrimidin-5-yl)benzene-1-sulfonyl]piperazin-1-yl}ethyl 6-(nitrooxy)hexanoate (Compound (14))

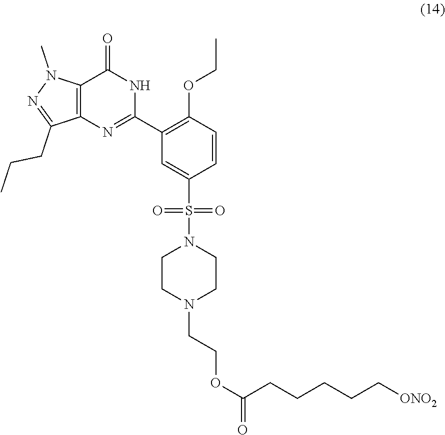

2-{4-[4-ethoxy-3-(1-methyl-7-oxo-3-propyl-6,7-dihydro-1H-pyrazolo[4,3-d] pyrimidin-5-yl)benzene-1-sulfonyl]piperazin-1-yl}ethyl 3-[(2S)-2,3-bis(nitrooxy) propoxy]propanoate (Compound (15))

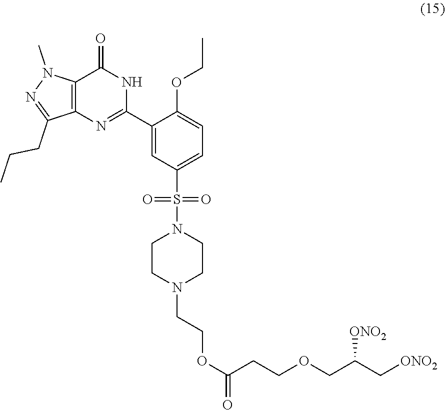

Most preferred compounds of formula (I) for use in the method for the treatment of dry age-related macular degeneration and or geographic atrophy are selected from the group consisting of:

2-{4-[3-(5-ethyl-4-oxo-7-propyl-4,5-dihydro-3H-pyrrolo [3,2-d] pyrimidin-2-yl)-4-propoxybenzene-1-sulfonyl]piperazin-1-yl}ethyl 3-[(2S)-2,3-bis(nitrooxy)propoxy] propanoate (Compound (1));

2-hydroxypropane-1,2,3-tricarboxylic acid 2-{4-[3-(5-ethyl-4-oxo-7-propyl-4,5-dihydro-3H-pyrrolo[3,2-d]pyrimidin-2-yl)-4-propoxybenzene-1-sulfonyl]piperazin-1-yl}ethyl 3-[(2S)-2,3-bis(nitrooxy)propoxy]propanoate (1/1) (citrate salt of Compound 1);

2-{4-[3-(5-ethyl-4-oxo-7-propyl-4,5-dihydro-3H-pyrrolo[3,2-d]pyrimidin-2-yl)-4-propoxybenzene-1-sulfonyl]piperazin-1-yl}ethyl 3-[(2S)-2,3-bis(nitrooxy)propoxy] propanoate hydrogen chloride (hydrochloride salt of Compound (1)).

Most preferred compounds of formula (II) for use in the method for the treatment of dry age-related macular degeneration and/or geographic atrophy are selected from the group consisting of:

[(2S)-1-(4-{[(3-Chloro-4-methoxyphenyl)methyl]amino}-5-{[(pyrimidin-2-yl)methyl]carbamoyl}pyrimidin-2-yl)pyrrolidin-2-yl]methyl 6-(nitrooxy)hexanoate (Compound (7));

[(2S)-1-(4-{[(3-chloro-4-methoxyphenyl)methyl]amino}-5-{[(pyrimidin-2-yl)methyl]carbamoyl}pyrimidin-2-yl)pyrrolidin-2-yl]methyl 3-[(2S)-2,3-bis(nitrooxy) propoxy]propanoate (Compound (10)).

Another embodiment of the invention relates to the use of a compound of formula (I) or a pharmaceutically acceptable salt thereof in a method of providing neuroprotection to a patient suffering from glaucoma or in a method of providing neuroprotection to a patient suffering from glaucoma, wherein said compound is selected from the group consisting of:

2-(4-(3-(5-ethyl-4-oxo-7-propyl-4,5-dihydro-3H-pyrrolo[3,2-d]pyrimidin-2-yl)-4-propoxyphenylsulfonyl)piperazin-1-yl)ethyl 3-[(2S)-2,3-bis(nitrooxy)propoxy]propanoate (Compound (1))

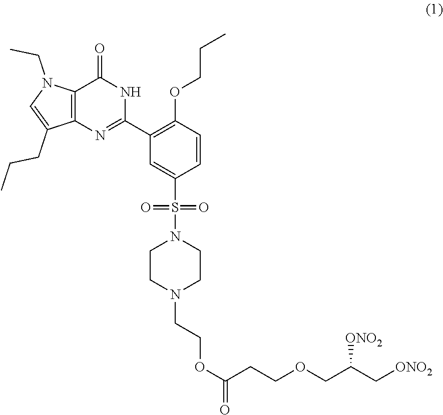

2-(4-((3-(5-ethyl-4-oxo-7-propyl-4,5-dihydro-3H-pyrrolo[3,2-d]pyrimidin-2-yl)-4-propoxyphenyl)sulfonyl)piperazin-1-yl)ethyl 2-(2-(nitrooxy)ethoxy)acetate (Compound (2))

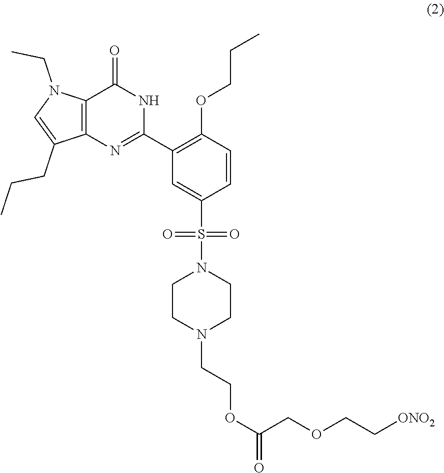

2-(4-(3-(5-ethyl-4-oxo-7-propyl-4,5-dihydro-3H-pyrrolo[3,2-d]pyrimidin-2-yl)-4-propoxyphenylsulfonyl)piperazin-1-yl)ethyl (5S)-5,6-bis(nitrooxy)hexanoate (Compound (3))

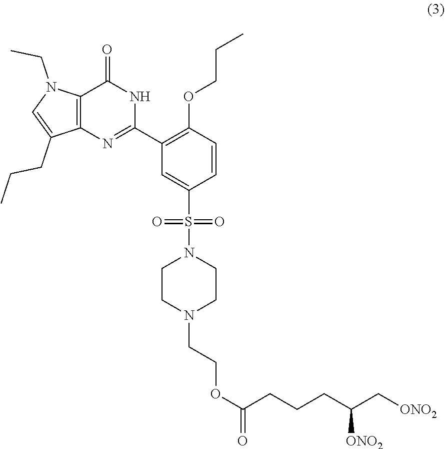

2-(4-((3-(5-ethyl-4-oxo-7-propyl-4,5-dihydro-3H-pyrrolo[3,2-d]pyrimidin-2-yl)-4-propoxyphenyl)sulfonyl)piperazin-1-yl)ethyl 6-(nitrooxy)hexanoate (Compound (4))

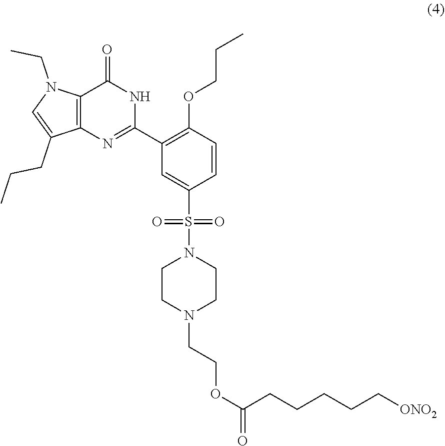

2-(4-(3-(5-ethyl-4-oxo-7-propyl-4,5-dihydro-3H-pyrrolo[3,2-d]pyrimidin-2-yl)-4-propoxyphenylsulfonyl)piperazin-1-yl)ethyl 6-(nitrooxy)hexyl carbonate (Compound (5))

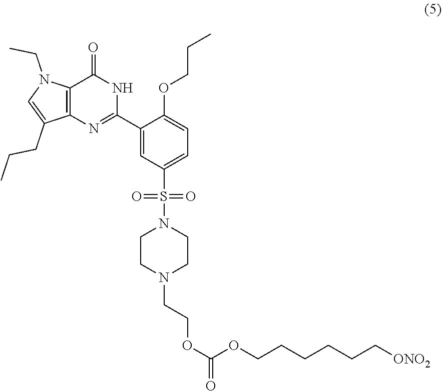

2-(4-(3-(5-ethyl-4-oxo-7-propyl-4,5-dihydro-3H-pyrrolo [3,2-d]pyrimidin-2-yl)-4-propoxyphenylsulfonyl)piperazin-1-yl)ethyl (5S)-5,6-bis(nitrooxy)hexyl carbonate (Compound (6))

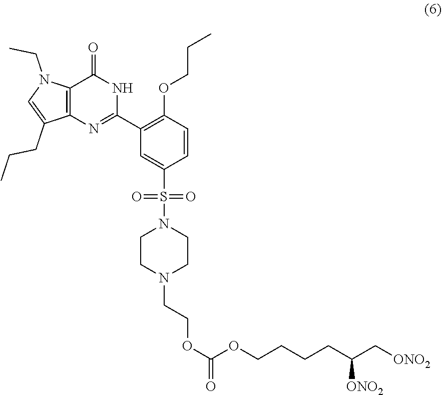

Another embodiment of the invention relates to the use a compound of formula (II) or a pharmaceutically acceptable salt thereof in a method of providing neuroprotection to a patient suffering from glaucoma or in a method of treating or preventing retinal neuropathies, wherein said compound is selected from the group consisting of:

[(2S)-1-(4-{[(3-chloro-4-methoxyphenyl)methyl]amino}-5-{[(pyrimidin-2-yl) methyl]carbamoyl}pyrimidin-2-yl)pyrrolidin-2-yl]methyl 6-(nitrooxy)hexanoate (Compound (7))

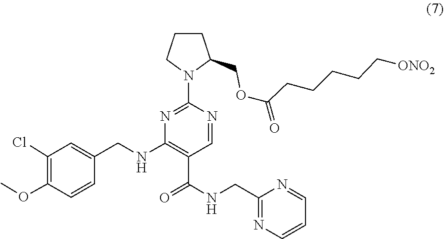

[(2S)-1-(4-{[(3-chloro-4-methoxyphenyl)methyl]amino}-5-{[(pyrimidin-2-yl) methyl]carbamoyl}pyrimidin-2-yl)pyrrolidin-2-yl]methyl (5S)-5,6-bis(nitrooxy) hexanoate (Compound (8))

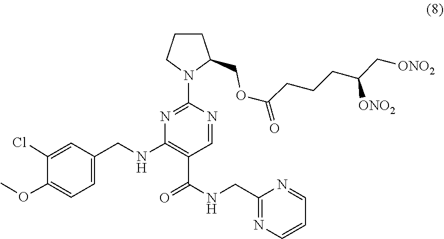

(S)-(1-(4-(3-chloro-4-methoxybenzylamino)-5-(pyrimidin-2-ylmethylcarbamoyl) pyrimidin-2-yl)pyrrolidin-2-yl)methyl 2-(2-(nitrooxy)ethoxy)acetate (Compound (9))

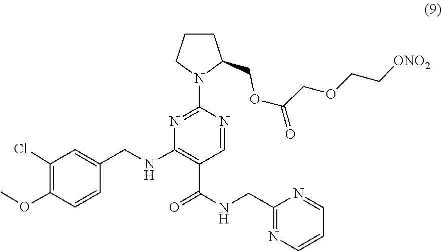

[(2S)-1-(4-{[(3-chloro-4-methoxyphenyl)methyl]amino}-5-{[(pyrimidin-2-yl) methyl]carbamoyl}pyrimidin-2-yl)pyrrolidin-2-yl]methyl 3-[(2S)-2,3-bis(nitrooxy) propoxy]propanoate (Compound (10))

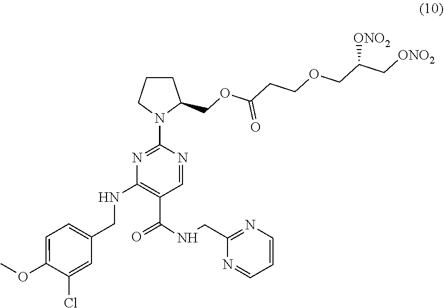

(S)-(1-(4-(3-chloro-4-methoxybenzylamino)-5-(pyrimidin-2-ylmethylcarbamoyl) pyrimidin-2-yl)pyrrolidin-2-yl)methyl 6-(nitrooxy)hexyl carbonate (Compound (11))

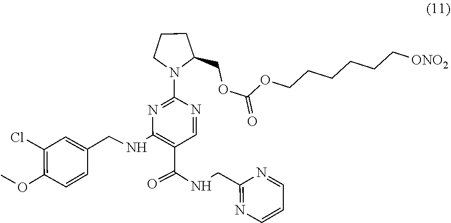

((S)-1-(4-(3-chloro-4-methoxybenzylamino)-5-(pyrimidin-2-ylmethylcarbamoyl) pyrimidin-2-yl)pyrrolidin-2-yl)methyl (5S)-5,6-bis(nitrooxy)hexyl carbonate (Compound (12))

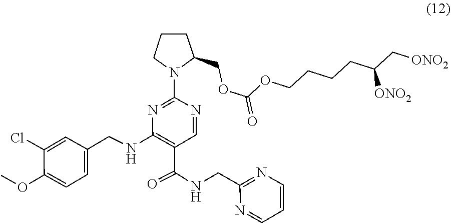

Another embodiment of the invention provides the use of a compound of formula (III) or a pharmaceutically acceptable salt thereof in a method of providing neuroprotection to a patient suffering from glaucoma or in a method of treating or preventing retinal neuropathies, wherein said compound is selected from the group consisting of:

2-{4-[4-ethoxy-3-(1-methyl-7-oxo-3-propyl-6,7-dihydro-1H-pyrazolo[4,3-d] pyrimidin-5-yl)benzene-1-sulfonyl]piperazin-1-yl}ethyl (5S)-5,6-bis(nitrooxy)hexanoate (Compound (13))

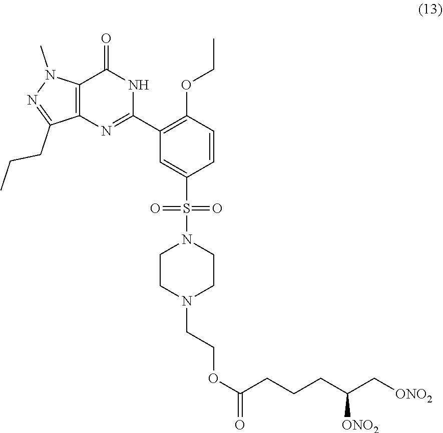

2-{4-[4-ethoxy-3-(1-methyl-7-oxo-3-propyl-6,7-dihydro-1H-pyrazolo[4,3-d] pyrimidin-5-yl)benzene-1-sulfonyl]piperazin-1-yl}ethyl 6-(nitrooxy)hexanoate (Compound (14))

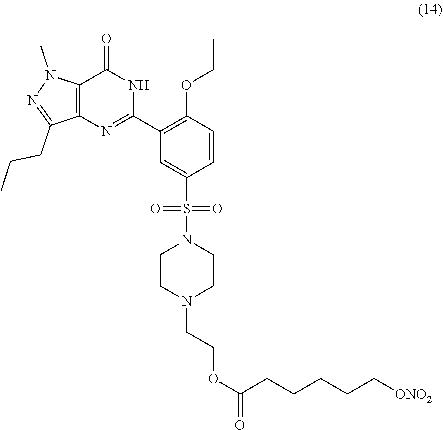

2-{4-[4-ethoxy-3-(1-methyl-7-oxo-3-propyl-6,7-dihydro-1H-pyrazolo[4,3-d] pyrimidin-5-yl)benzene-1-sulfonyl]piperazin-1-yl}ethyl 3-[(2S)-2,3-bis(nitrooxy) propoxy]propanoate (Compound (15))

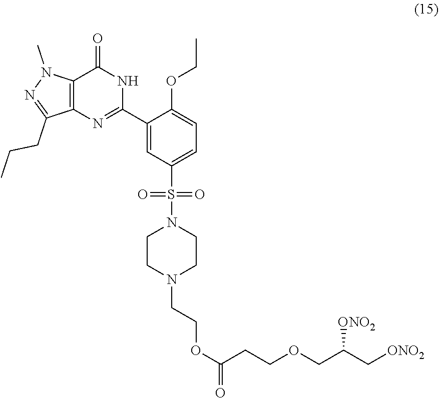

Most preferred compounds of formula (I) for use in the method of providing neuroprotection to a patient suffering from glaucoma or in a method of treating or preventing retinal neuropathies are selected from the group consisting of:

2-{4-[3-(5-ethyl-4-oxo-7-propyl-4,5-dihydro-3H-pyrrolo[3,2-d]pyrimidin-2-yl)-4-propoxybenzene-1-sulfonyl]piperazin-1-yl}ethyl 3-[(2S)-2,3-bis(nitrooxy)propoxy] propanoate (Compound (1));

2-hydroxypropane-1,2,3-tricarboxylic acid 2-{4-[3-(5-ethyl-4-oxo-7-propyl-4,5-dihydro-3H-pyrrolo[3,2-d]pyrimidin-2-yl)-4-propoxybenzene-1-sulfonyl]piperazin-1-yl}ethyl 3-[(2S)-2,3-bis(nitrooxy)propoxy]propanoate (1/1) (citrate salt of Compound 1);

2-{4-[3-(5-ethyl-4-oxo-7-propyl-4,5-dihydro-3H-pyrrolo[3,2-d]pyrimidin-2-yl)-4-propoxybenzene-1-sulfonyl]piperazin-1-yl}ethyl 3-[(2S)-2,3-bis(nitrooxy)propoxy] propanoate hydrogen chloride (hydrochloride salt of Compound (1)).

Most preferred compounds of formula (II) for use in the method of providing neuroprotection to a patient suffering from glaucoma are selected from the group consisting of:

[(2S)-1-(4-{[(3-Chloro-4-methoxyphenyl)methyl]amino}-5-{[(pyrimidin-2-yl)methyl]carbamoyl}pyrimidin-2-yl)pyrrolidin-2-yl]methyl 6-(nitrooxy)hexanoate (Compound (7));

[(2S)-1-(4-{[(3-chloro-4-methoxyphenyl)methyl]amino}-5-{[(pyrimidin-2-yl)methyl]carbamoyl}pyrimidin-2-yl)pyrrolidin-2-yl]methyl 3-[(2S)-2,3-bis(nitrooxy) propoxy]propanoate (Compound (10)).

Another embodiment of the invention provides a method of treating dry age-related macular degeneration and/or geographic atrophy which comprises administering to a patient in need of such treatment a therapeutically effective amount of an ophthalmic formulation comprising a compound of formula (I), or formula (II), or formula (III) or a stereoisomer or a pharmaceutically acceptable salt thereof and one or more pharmaceutically acceptable excipients and/or an ophthalmically acceptable vehicle.

Another embodiment of the invention provides a method of providing neuroprotection to a patient suffering from glaucoma or a method of treating or preventing retinal neuropathies which comprises administering to a patient in need of such treatment a therapeutically effective amount of an ophthalmic formulation comprising a compound of formula (I), or formula (II), or formula (III) or a stereoisomer or a pharmaceutically acceptable salt thereof and one or more pharmaceutically acceptable excipients and/or an ophthalmically acceptable vehicle.

The compounds of the invention can also be administered in the form of ophthalmic pharmaceutical compositions that are formulated as solution, suspension, emulsions, hydrogel or as sustained-release ophthalmic drug delivery system (posterior segment drug delivery system) to provide long term treatment.

The compounds of the invention are administered locally to the eye, preferably the compounds are administered by intraocular injection such as intravitreal injection, or periorbital injection such as subtenon injection. The compounds of the invention can also be formulated and administered in the form of sustained-release intravitreal implants.

For periorbital injection the ug/eye of the compounds of the invention dissolved into the ophthalmic pharmaceutical composition is generally from 1 to 1000 μg/eye, preferably ranging from 3 to 300 μg/eye and, most preferably included between 10 and 100 μg/eye. Likewise, for intravitreal injection the μg/eye of the compounds of the invention included into the ophthalmic pharmaceutical matrix is generally from 1 to 1000 μg/eye, preferably from 3 to 300 μg/eye and, most preferably included from 10 to 100 μg/eye.

The actual dose and frequency of the administration of the compounds of the present invention depend on the specific compound, and on the condition to be treated.

As used herein, treating a disease also includes slowing its progress and or relieving the disease, e.g., causing regression of the disease. In some embodiments, the progressive worsening (e.g., the increasing intensity) of a symptom is slowed, reduced, or halted, for example retinal pigment epithelium cell death is reduced or the size of geographic atrophy is reduced. Treating dry-AMD includes preventing or delaying vision loss or the progression of dry-AMD to the advanced stage of the disease also known as geographic atrophy. Treating geographic atrophy includes inhibiting progression of geographic atrophy by reducing the atrophic lesions of the outer retina and of the retinal pigment epithelium.

As used herein, providing “neuroprotection to a patient suffering from glaucoma” includes preventing or delaying degeneration of retinal ganglion cells (RGCs) and axons in the optic nerve.

As further used herein, providing “neuroprotection to a patient suffering from retinal neuropathies” includes preventing or delaying loss of vision or deterioration of vision.

“Pharmaceutically acceptable excipient” refers to a substance that aids the administration of an active agent to a subject and can be included in the pharmaceutical composition without causing adverse toxicological effect on the subject.

The term “ophthalmically acceptable vehicle” means a pharmaceutical composition having physical properties (e.g., pH and/or osmolality) that are physiologically compatible with ophthalmic tissues for intravitreal and other ophthalmic administrations.

The compounds of formula (I), formula (II) or formula (III) and stereoisomers or salts thereof of the invention can be prepared according to the methods of synthesis disclosed in WO 2020/030489.

1. A method for treating dry age-related macular degeneration and/or geographic atrophy, providing neuroprotection to a patient suffering from glaucoma or treating or preventing retinal neuropathies, comprising administering a compound of formula (I), formula (II) or formula (III) or a stereoisomer or a pharmaceutically acceptable salt thereof 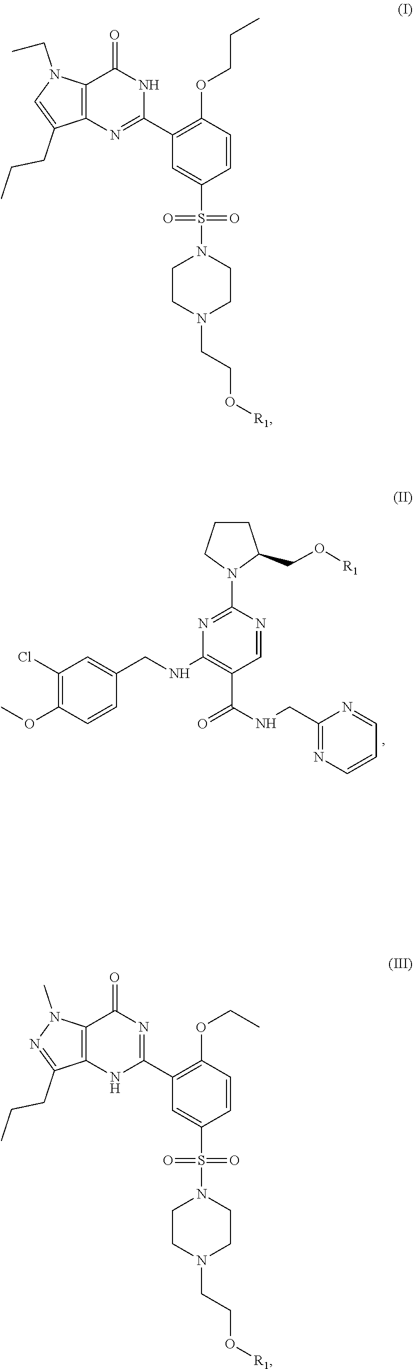
wherein:
R₁ is the residue of a nitric oxide releasing molecule having the following formula:
R₁=—C(O)—(O—CH₂)_{y}(CH₂)ₘ—[O—(CH₂)ₙ]ₚ—(CH—ONO₂)_{q}—CH₂—ONO₂
wherein:
y is 1 or 0;
p is 1 or 0;
q is 1 or 0;
m is an integer ranging from 1 to 10; and
n is an integer ranging from 1 to 6.

2. The method according to claim 1, wherein y is 0.

3. The method according to claim 1, wherein R₁ is selected from the group consisting of:

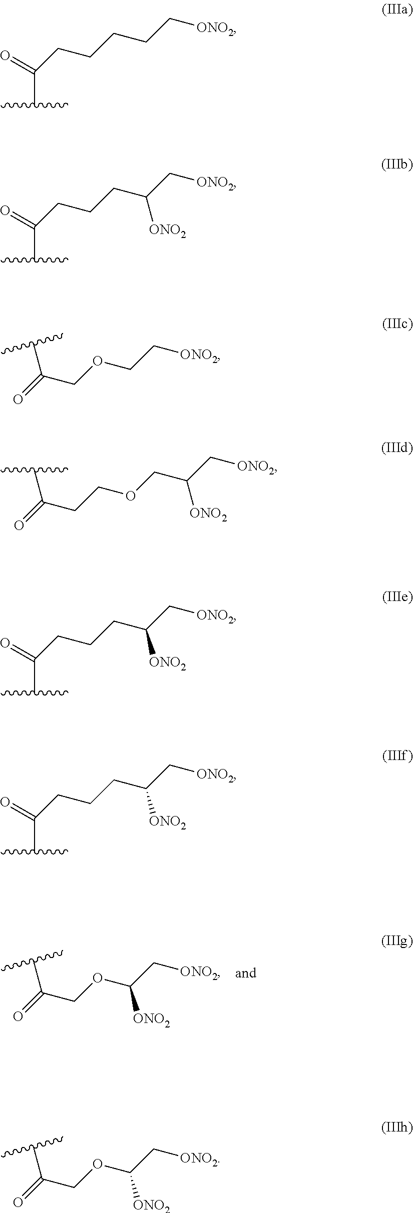

4. The method according to claim 1, wherein R₁ is selected from the group consisting of:

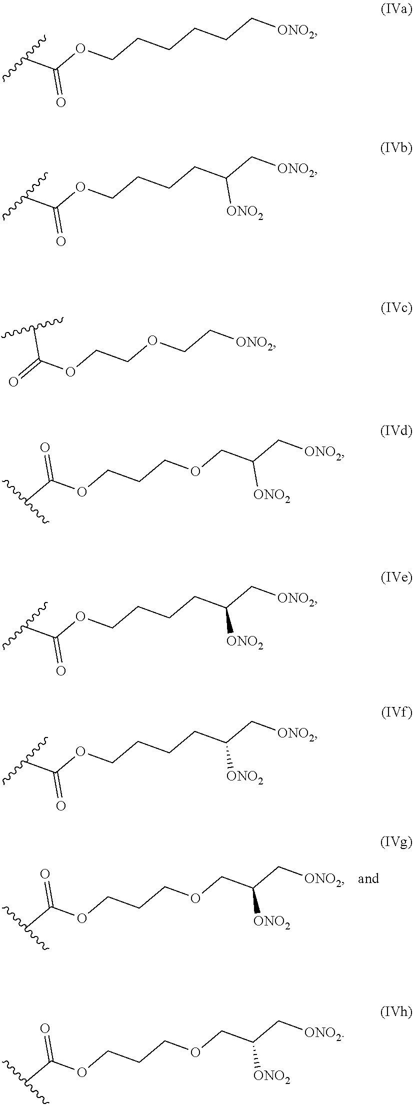

5. The method according to claim 3, wherein the compound is a compound of formula (I) and R₁ is (IIIg) or (IIIh).

6. The method according to claim 3, wherein the compound is a compound of formula (II) and R₁ is selected from (IIIa), (IIIg) or (IIIh).

7. The method according to claim 3, wherein the compound is a compound of formula (III) and R₁ is selected from (IIIa), (IIIg) or (IIIh).

8. The method according to claim 3, wherein the compound is selected from the group consisting of:
2-(4-(3-(5-ethyl-4-oxo-7-propyl-4,5-dihydro-3H-pyrrolo[3,2-d]pyrimidin-2-yl)-4-propoxyphenylsulfonyl)piperazin-1-yl)ethyl 3-[(2S)-2,3-bis(nitrooxy)propoxy]propanoate (Compound (1)) 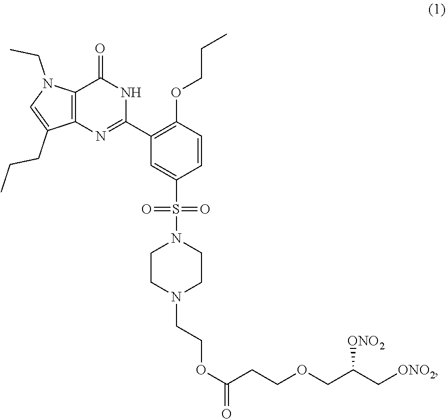
2-(4-((3-(5-ethyl-4-oxo-7-propyl-4,5-dihydro-3H-pyrrolo[3,2-d]pyrimidin-2-yl)-4-propoxyphenyl)sulfonyl)piperazin-1-yl)ethyl 2-(2-(nitrooxy)ethoxy)acetate (Compound (2)) 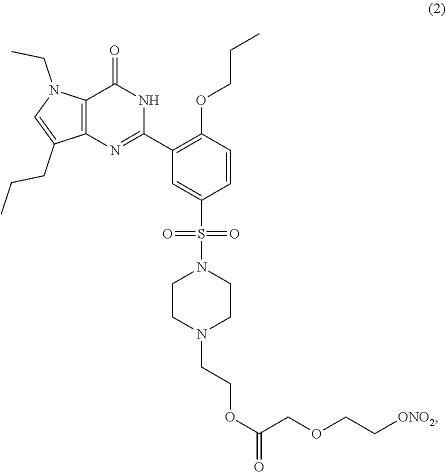
2-(4-(3-(5-ethyl-4-oxo-7-propyl-4,5-dihydro-3H-pyrrolo[3,2-d]pyrimidin-2-yl)-4-propoxyphenylsulfonyl)piperazin-1-yl)ethyl (5S)-5,6-bis(nitrooxy)hexanoate (Compound (3)) 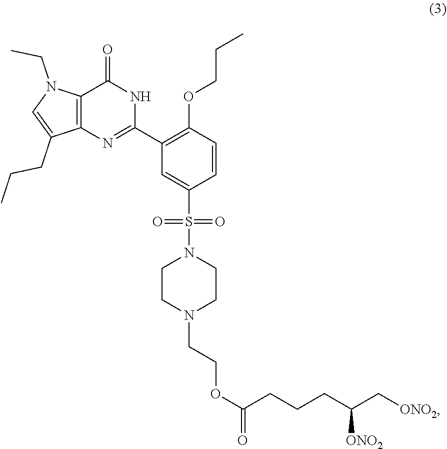
2-(4-((3-(5-ethyl-4-oxo-7-propyl-4,5-dihydro-3H-pyrrolo[3,2-d]pyrimidin-2-yl)-4-propoxyphenyl)sulfonyl)piperazin-1-yl)ethyl 6-(nitrooxy)hexanoate (Compound (4)) 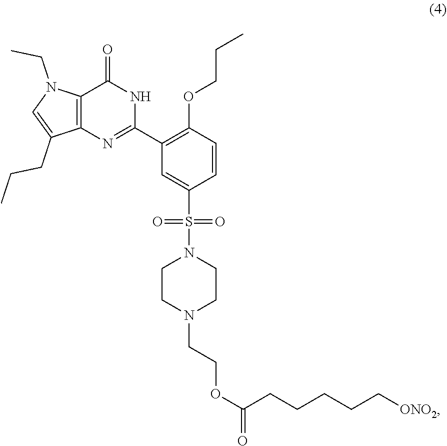
[(2S)-1-(4-{[(3-chloro-4-methoxyphenyl)methyl]amino}-5-{[(pyrimidin-2-yl) methyl]carbamoyl}pyrimidin-2-yl)pyrrolidin-2-yl]methyl 6-(nitrooxy)hexanoate (Compound (7)) 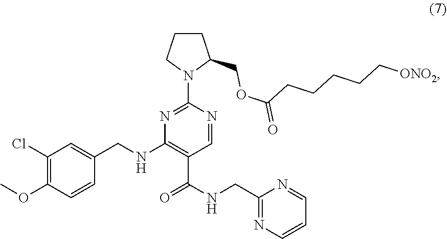
[(2S)-1-(4-{[(3-chloro-4-methoxyphenyl)methyl]amino}-5-{[(pyrimidin-2-yl) methyl]carbamoyl}pyrimidin-2-yl)pyrrolidin-2-yl]methyl (5S)-5,6-bis(nitrooxy) hexanoate (Compound (8)) 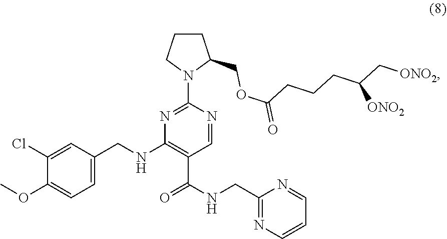
(S)-(1-(4-(3-chloro-4-methoxybenzylamino)-5-(pyrimidin-2-ylmethylcarbamoyl) pyrimidin-2-yl)pyrrolidin-2-yl)methyl 2-(2-(nitrooxy)ethoxy)acetate (Compound (9)) 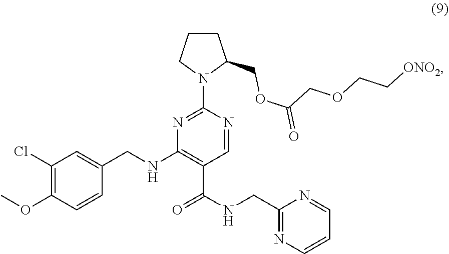
[(2S)-1-(4-{[(3-chloro-4-methoxyphenyl)methyl]amino}-5-{[(pyrimidin-2-yl) methyl]carbamoyl}pyrimidin-2-yl)pyrrolidin-2-yl]methyl 3-[(2S)-2,3-bis(nitrooxy) propoxy]propanoate (Compound (10)) 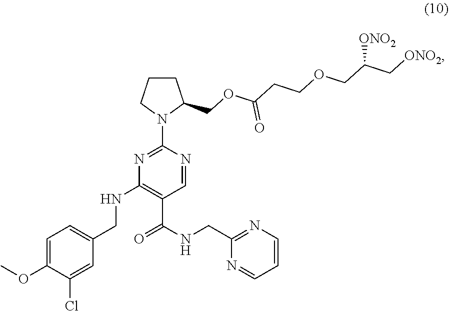
2-{4-[4-ethoxy-3-(1-methyl-7-oxo-3-propyl-6,7-dihydro-1H-pyrazolo[4,3-d] pyrimidin-5-yl)benzene-1-sulfonyl]piperazin-1-yl}ethyl (5S)-5,6-bis(nitrooxy)hexanoate (Compound (13)) 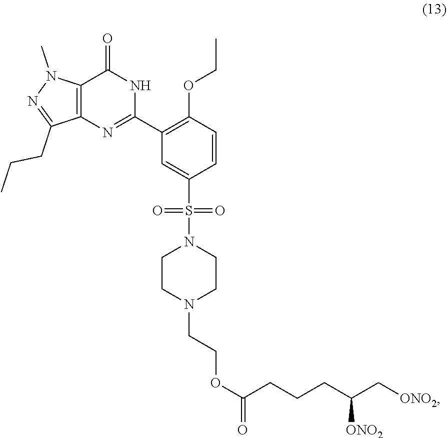
2-{4-[4-ethoxy-3-(1-methyl-7-oxo-3-propyl-6,7-dihydro-1H-pyrazolo[4,3-d] pyrimidin-5-yl)benzene-1-sulfonyl]piperazin-1-yl}ethyl 6-(nitrooxy)hexanoate (Compound (14)) 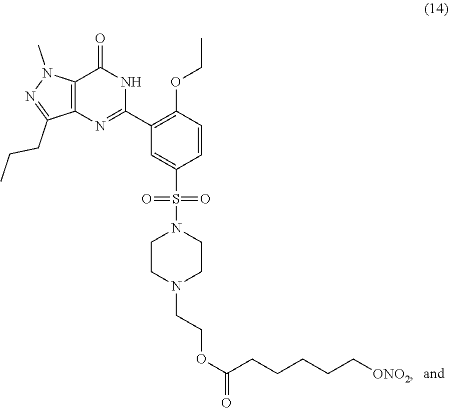
and
2-{4-[4-ethoxy-3-(1-methyl-7-oxo-3-propyl-6,7-dihydro-1H-pyrazolo[4,3-d] pyrimidin-5-yl)benzene-1-sulfonyl]piperazin-1-yl}ethyl 3-[(2S)-2,3-bis(nitrooxy) propoxy] propanoate (Compound 15)

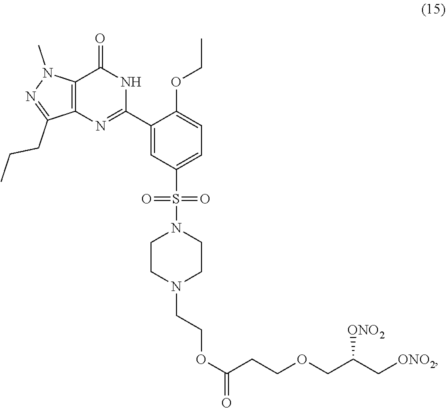

9. The method according to claim 3, wherein the compound is selected from the group consisting of:
2-{4-[3-(5-ethyl-4-oxo-7-propyl-4,5-dihydro-3H-pyrrolo[3,2-d]pyrimidin-2-yl)-4-propoxybenzene-1-sulfonyl]piperazin-1-yl}ethyl 3-[(2S)-2,3-bis(nitrooxy)propoxy] propanoate (Compound (1));
2-hydroxypropane-1,2,3-tricarboxylic acid 2-{4-[3-(5-ethyl-4-oxo-7-propyl-4,5-dihydro-3H-pyrrolo[3,2-d]pyrimidin-2-yl)-4-propoxybenzene-1-sulfonyl]piperazin-1-yl}ethyl 3-[(2S)-2,3-bis(nitrooxy)propoxy]propanoate (1/1) (citrate salt of Compound (1));
2-{4-[3-(5-ethyl-4-oxo-7-propyl-4,5-dihydro-3H-pyrrolo[3,2-d]pyrimidin-2-yl)-4-propoxybenzene-1-sulfonyl]piperazin-1-yl}ethyl 3-[(2S)-2,3-bis(nitrooxy)propoxy] propanoate hydrogen chloride (hydrochloride salt of Compound (1));
[(2S)-1-(4-{[(3-Chloro-4-methoxyphenyl)methyl]amino}-5-{[(pyrimidin-2-yl)methyl]carbamoyl}pyrimidin-2-yl)pyrrolidin-2-yl]methyl 6-(nitrooxy)hexanoate (Compound (7)); and
[(2S)-1-(4-{[(3-chloro-4-methoxyphenyl)methyl]amino}-5-{[(pyrimidin-2-yl)methyl]carbamoyl}pyrimidin-2-yl)pyrrolidin-2-yl]methyl 3-[(2S)-2,3-bis(nitrooxy) propoxy]propanoate (Compound (10)).

10. The method according to claim 4, wherein the compound is selected from the group consisting of:
2-(4-(3-(5-ethyl-4-oxo-7-propyl-4,5-dihydro-3H-pyrrolo[3,2-d]pyrimidin-2-yl)-4-propoxyphenylsulfonyl)piperazin-1-yl)ethyl 6-(nitrooxy)hexyl carbonate (Compound (5)) 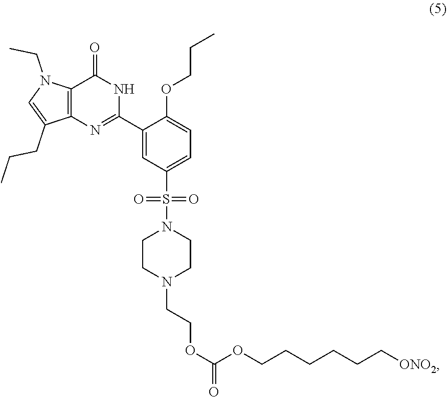
2-(4-(3-(5-ethyl-4-oxo-7-propyl-4,5-dihydro-3H-pyrrolo[3,2-d]pyrimidin-2-yl)-4-propoxyphenylsulfonyl)piperazin-1-yl)ethyl (5S)-5,6-bis(nitrooxy)hexyl carbonate (Compound (6)) 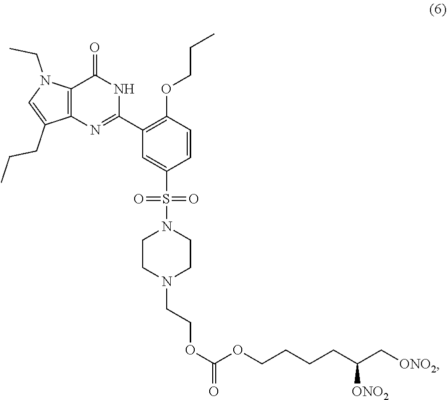
(S)-(1-(4-(3-chloro-4-methoxybenzylamino)-5-(pyrimidin-2-ylmethylcarbamoyl) pyrimidin-2-yl)pyrrolidin-2-yl)methyl 6-(nitrooxy)hexyl carbonate (Compound (11)) 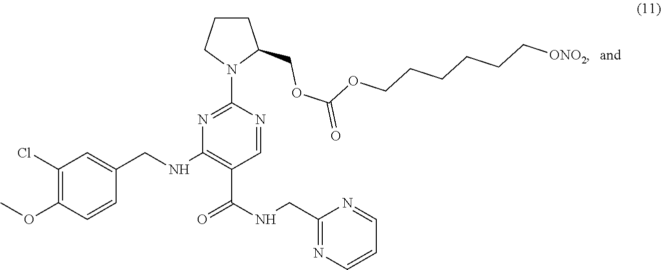
and
((S)-1-(4-(3-chloro-4-methoxybenzylamino)-5-(pyrimidin-2-ylmethylcarbamoyl) pyrimidin-2-yl)pyrrolidin-2-yl)methyl (5S)-5,6-bis(nitrooxy)hexyl carbonate (Compound (12))

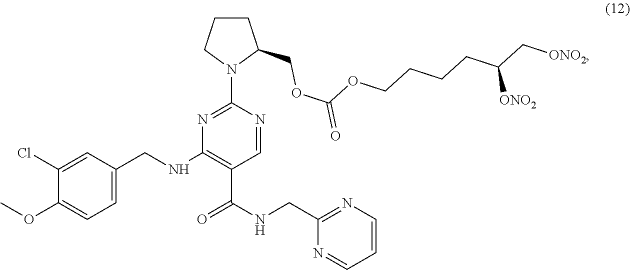

11. The method according to claim 1, comprising a compound of formula (III) wherein the compound is [(2S)-1-(4-{[(3-Chloro-4-methoxyphenyl)methyl]amino}-5-{[(pyrimidin-2-yl)methyl] carbamoyl}pyrimidin-2-yl)pyrrolidin-2-yl]methyl 6-(nitrooxy)hexanoate (Compound (7)).

12. The method according to claim 1, comprising a compound of formula (I), formula (II) or formula (III) wherein the compound is administered locally to the eye, by ocular injection such as intravitreal injection, or periorbital injection such as subtenon injection.

13. The method according to claim 1, comprising a compound of formula (I), formula (II) or formula (III), wherein the compound is formulated as an ophthalmic formulation comprising a compound of formula (I), or formula (II), or formula (III) and one or more pharmaceutically acceptable excipients and/or an ophthalmically acceptable vehicle.

14. The method according to claim 13, comprising a compound of formula (I), formula (II) or formula (III), wherein the ophthalmic formulation is in the form of solution, suspension, emulsions, hydrogel, sustained-release ophthalmic drug delivery system or intravitreal implant.
